(19) United States
(12) Patent Application Publication
(10) Pub. No.: US 2005/0273947 A1
(43) Pub. Date: Dec. 15, 2005

(54) Composition for dyeing keratin fibers comprising at least one compound bearing at least one amine function, at least one pigment and at least one chemical coupling agent

(76) Inventor: Gaelle Brun, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/140,453

(22) Filed: May 31, 2005

Related U.S. Application Data
(60) Provisional application No. 60/580,101, filed on Jun. 17, 2004.

(30) Foreign Application Priority Data
May 28, 2004 (FR) 04 05842

(51) Int. Cl. A61K 7/13

(52) U.S. Cl. 8/405

(57) ABSTRACT

The present invention relates to a composition for dyeing keratin fibers comprising at least one compound bearing at least one amine function, at least one pigment and at least one chemical coupling agent, to the processes for dyeing keratin fibers, using this compound bearing at least one amine function, this pigment and this chemical coupling agent, and also to the use of this compound bearing at least one amine function, of this pigment and of this chemical coupling agent for dyeing keratin fibers. In at least one embodiment, the present invention allows the production of a coloration that is visible on a dark support without it being necessary to lighten or bleach the keratin fibers, and that shows good resistance to the various attacking factors to which the hair may be subjected, in particular to shampoos and to rubbing.

This application claims benefit of U.S. Provisional Application No. 60/580,101, filed Jun. 17, 2004, the contents of which are incorporated herein by reference. This application also claims benefit of priority under 35 U.S.C. § 119 to French Patent Application No. 04 05842, filed May 28, 2004, the contents of which are also incorporated by reference.

The present disclosure relates to a composition for dyeing keratin fibers, for example, human keratin fibers such as the hair, comprising at least one compound bearing at least one amine function, at least one pigment other than iron oxide, titanium oxide and silica, and at least one chemical coupling agent for forming amide bonds.

In the field of dyeing keratin fibers, there are two main modes of dyeing, each of which have their advantages and their drawbacks.

Direct dyeing or semi-permanent dyeing comprises dyeing keratin fibers with dye compositions comprising direct dyes. These dyes are colored and coloring molecules that have an affinity for keratin fibers. They are usually applied to the keratin fibers for a time necessary to obtain the desired coloration, and are then rinsed out.

The standard dyes that are used include, for example, dyes of the nitrobenzene, anthraquinone, nitropyridine, azo, xanthene, acridine, azine or triarylmethane type or natural dyes.

It has also been proposed to use pigments, for instance in patent application FR 2 741 530, which envisages the use for dyeing keratin fibers of a composition comprising at least one dispersion of particles of film-forming polymer comprising at least one acid function and at least one pigment dispersed in the continuous phase of the dispersion.

The colorations obtained via this mode of dyeing (direct or semi-permanent) may have the drawback of having low shampoo-fastness.

Oxidation dyeing or permanent dyeing comprises dyeing keratin fibers with dye compositions comprising oxidation dye precursors, also known, for example, as oxidation bases, such as ortho- or para-phenylenediamines, ortho- or para-aminophenols and heterocyclic compounds such as diaminopyrazole derivatives. These oxidation bases may be colorless or weakly colored compounds, which, when combined with oxidizing products, for example, can give rise to colored compounds via a process of oxidative condensation.

It is also known that the shades obtained with these oxidation bases can be varied by combining them with couplers or coloration modifiers, the latter being chosen, for example, from aromatic meta-diamines, meta-aminophenols, meta-diphenols and certain heterocyclic compounds such as indole compounds.

The shades obtained via oxidation dyeing generally show relatively good shampoo-fastness.

One potential difficulty with many examples of these two modes of dyeing, direct dyeing or semi-permanent dyeing and oxidation dyeing or permanent dyeing, is that if it is desired to dye a dark support, it may be necessary to bleach or lighten the support in order for the supplied color to be visible.

The bleaching or lightening of keratin fibers may be performed using an oxidizing agent. Among the conventionally used oxidizing agents that may be mentioned are hydrogen peroxide or compounds capable of producing hydrogen peroxide by hydrolysis, for instance urea peroxide or persalts such as perborates, persulfates and percarbonates.

The use of oxidizing agents in some compositions, whether to lighten and/or to oxidize oxidation dyes, may have the drawback of resulting in appreciable degradation of the keratin fibers and/or of impairing their cosmetic properties. The hair may have a tendency to become coarse, more difficult to disentangle and more brittle.

Thus, it would be desirable to provide novel compositions for dyeing keratin fibers, for instance human keratin fibers such as the hair, which may allow, in some embodiments, colorations to be obtained that are visible on a dark support without it being necessary to lighten or bleach the fibers, and/or that show good shampoo-fastness.

Such compositions are disclosed herein. More specifically, one composition of the present disclosure is a composition, for example for dyeing keratin fibers, comprising, optionally, in a cosmetically acceptable medium:
at least one compound bearing at least one amine function;
at least one pigment other than iron oxide, titanium oxide and silica; and
at least one chemical coupling agent for forming amide bonds.

The composition in accordance with the present disclosure may lead to a visible coloration on a dark support without it being necessary to lighten or bleach the keratin fibers, and consequently without any physical degradation of the keratin fibers. This coloration may furthermore be resistant to at least one of the various attacking factors to which the hair may be subjected, such as shampoos, rubbing, light, bad weather, sweat and permanent reshaping operations. In at least one embodidment, the coloration may be resistant to shampoos and/or rubbing. The coloration obtained with the composition of the present disclosure may be in varied shades and be chromatic, strong, aesthetic and/or sparingly selective.

The present disclosure also relates to a process for dyeing keratin fibers using at least one compound bearing at least one amine function, at least one pigment other than iron oxide, titanium oxide and silica, and at least one chemical coupling agent for forming amide bonds.

The present disclosure also relates to the use for dyeing keratin fibers of at least one compound bearing at least one amine function, at least one pigment other than iron oxide, titanium oxide and silica, and at least one chemical coupling agent for forming amide bonds.

According to one embodiment of the present disclosure, the at least one compound bearing at least one amine function has a molecular weight of greater than 300 g.mol⁻¹. For example, the at least one compound bearing at least one amine function may have a molecular weight of greater than 1000 g.mol⁻¹. These compounds may be, for example, polymers.

In at least one embodiment, the polymers bearing at least one amine function are polyamines. Non-limiting examples of polyamines that may be mentioned include polyvinylamine, polyethyleneamine, polylysine, chitosan, polyallylamine, aminodextran, aminocellulose and aminopolyvinyl acetal.

As used herein, the term “pigment” means any organic and/or mineral species whose solubility in water is less than 0.01% at 20° C., having an absorption ranging from 350 to 700 nm, and in at least one embodiment, having an absorption with a maximum.

The pigments in accordance with the present disclosure may be chosen from all organic and/or mineral pigments, with the exception of iron oxide, titanium oxide and silica; see, for example, those described in Kirk-Othmer's Encyclopaedia of Chemical Technology and in Ullmann's Encyclopaedia of Industrial Chemistry.

The pigments in accordance with the present disclosure may be in the form of powder or of pigmentary paste. They may be coated or uncoated.

The pigments in accordance with the present disclosure may be chosen, for example, from white or colored pigments, lakes, pigments with special effects such as nacres or flakes, and mixtures thereof.

Non-limiting examples of white or colored mineral pigments that may be mentioned include zirconium oxides or cerium oxides, chromium oxides, manganese violet, ultramarine blue, chromium hydrate and ferric blue.

Non-limiting examples of white or colored organic pigments that may be mentioned include nitroso, nitro, azo, xanthene, quinoline, anthraquinone and phthalo-cyanin compounds, compounds of metallic complex type, and isoindolinone, isoindoline, quinacridone, perinone, perylene, diketopyrrolopyrrole, thioindigo, dioxazine, triphenylmethane and quinophthalone compounds.

In at least one embodiment, the white or colored organic pigments may be chosen from carmine, carbon black, aniline black, azo yellow, quinacridone, phthalocyanin blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in patent FR 2 679 771.

Pigmentary pastes of organic pigment may also be used, such as the products sold by the company Hoechst under the name:
Jaune Cosmenyl IOG: Pigment Yellow 3 (CI 11710);
Jaune Cosmenyl G: Pigment Yellow 1 (CI 11680);
Orange Cosmenyl GR: Pigment Orange 43 (CI 71105);
Rouge Cosmenyl R: Pigment Red 4 (CI 12085);
Carmin Cosmenyl FB: Pigment Red 5 (CI 12490);
Violet Cosmenyl RL: Pigment Violet 23 (CI 51319);
Bleu Cosmenyl A2R: Pigment Blue 15.1 (CI 74160);
Vert Cosmenyl GG: Pigment Green 7 (CI 74260);
Noir Cosmenyl R: Pigment Black 7 (CI 77266).

The pigments in accordance with the present disclosure may also be in the form of composite pigments as described in patent EP 1 184 426. These composite pigments may, for instance, be composed of particles comprising:
a mineral core,
at least one binder for fixing the organic pigments to the core, and
at least one organic pigment at least partially covering the core.

As used herein, the term “lakes” means dyes adsorbed onto insoluble particles, the assembly thus obtained remaining insoluble during use. The mineral substrates onto which the dyes are adsorbed are, for example, alumina, silica, calcium sodium borosilicate or calcium aluminium borosilicate, and aluminium. Among the organic dyes that may be mentioned is cochineal carmine.

Non-limiting examples of lakes that may be mentioned include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), D & C Blue 1 (CI 42 090).

As used herein, the term “pigments with special effects” means pigments that can generally create a colored appearance (characterized by a certain shade, a certain vivacity and/or a certain level of luminance) that is non-uniform and that may change as a function of the conditions of observation (light, temperature, angles of observation, etc.). These pigments with special effects are consequently in contrast with white or colored pigments, which afford a standard opaque, semi-transparent or transparent uniform shade.

Non-limiting examples of pigments with special effects that may be mentioned include white nacreous pigments such as mica coated with titanium or with bismuth oxychloride, coloured nacreous pigments such as mica coated with titanium and with iron oxides, mica coated with titanium and, for example, with ferric blue or with chromium oxide, mica coated with titanium and with an organic pigment as defined above, and also nacreous pigments based on bismuth oxychloride.

Mention may also be made of pigments with an interference effect not bound to a substrate, for instance liquid crystals (Helicones HC from Wacker), holographic interference flakes (Geometric Pigments or Spectra f/x from Spectratek). The pigments with special effects also comprise fluorescent pigments, whether they are substances that are fluorescent in daylight or that produce ultraviolet fluorescence, phosphorescent pigments, photochromic pigments, thermochromic pigments and quantum dots, for example sold by the company Quantum Dots Corporation.

Quantum dots are luminescent semiconductive nanoparticles capable of emitting, under light excitation, radiation with a wavelength ranging from 400 nm to 700 nm. These nanoparticles are known in the literature. For example, they may be manufactured according to the processes described, for example, in U.S. Pat. No. 6,225,198 or U.S. Pat. No. 5,990,479, in the publications cited therein and also in the following publications: Dabboussi B. O. et al “(CdSe)ZnS core-shell quantum dots: synthesis and characterisation of a size series of highly luminescent nanocristallites” Journal of Physical Chemistry B, vol 101, 1997, pp 9463-9475 and Peng, Xiaogang et al, “Epitaxial Growth of highly Luminescent CdSe/CdS core/shell nanocrystals with photostability and electronic accessibility” Journal of the American Chemical Society, vol 119, No. 30, pp 7019-7029.

In at least one embodiment, the pigments disclosed herein may be colored pigments.

The variety of pigments used may make it possible to obtain a rich palette of colors, and also particular optical effects such as metallic or interference effects.

The size of a pigment other than the nacres in solution may generally range from 10 nm to 10 μm, such as from 50 nm to 5 μm and from 100 nm to 3 μm. The size of a nacre in solution may generally range from 1 to 200 μm, such as from 1 to 80 μm and from 1 to 50 μm.

According to one embodiment, the at least one pigment is in dispersion in the composition in accordance with the disclosure.

In the context of the present disclosure, the term “chemical coupling agent” means a chemical compound capable of increasing the rate of a reaction. For example, a chemical coupling agent for the formation of amide bonds is a chemical compound capable of promoting or accelerating the formation of amide bonds between one or more compounds. Non-limiting examples that may be mentioned include carbodiimide derivatives; 1,1′-carbonyldiimidazole; phosphonium salts; phosphonates; phosphoramides; dialkoxytriazine derivatives such as 4-(4,6-dimethoxy-1,3,5-triazin-2-yl)-4-methylmorpholinium chloride (DMT-MM) and 2-chloro-4,6-dimethoxy-1,3,5-triazine (CDMT).

According to one embodiment of the present disclosure, the carbodiimide derivatives are chosen from the compounds of formula (I) below:
R₁—N═C═N—R₂ (I)
wherein R₁ and R₂ are chosen from, independently of each other:
a hydrogen atom;
an alkyl radical, which is unsubstituted or substituted with at least one substituent chosen from:
   a hydroxyl radical;
   an amino radical;
   a monoalkylamino or dialkylamino radical;
   a mono- or di(mono- or polyhydroxyalkyl)amino radical;
   an aryl radical;
   a heterocyclic group;
an aryl radical; and
an arylalkyl radical.

In the context of the present disclosure, the term “alkyl radical” means a linear or branched, cyclic or acyclic radical containing from 1 to 30 carbon atoms, such as from 1 to 10 carbon atoms, for example methyl, ethyl, n-propyl, isopropyl and butyl radicals.

As used herein, the term “aryl radical” means a carbon-based radical derived from fused or non-fused benzene-based compounds, containing from 6 to 30 carbon atoms, for example phenyl, anthracenyl and naphthyl radicals.

As used herein, the term “heterocyclic group” means a fused or non-fused, aromatic or non-aromatic, 5- to 50-membered, such as 5- to 10-membered monocyclic or polycyclic group, for example a thiophene, benzofuran, benzothiophene, indole, bispyridine, benzopyran, quinoline, pyrazole, pyridine, pyrrole, furan, imidazole or benzimidazole ring.

In at least one embodiment, neither R₁ nor R₂ are not a hydrogen atom. In at least one embodiment, R₁ and R₂ are each independently an unsubstituted or substituted alkyl radical.

As examples of carbodiimide derivatives that are useful in the context of the disclosure, non-limiting mention may be made of N-(3-dimethylaminopropyl)-N′-(ethyl)carbodiimide; and N,N′-dicyclohexylcarbodiimide.

In at least one embodiment, the carbodiimide derivatives are soluble in water or in an alcoholic solvent.

The at least one chemical coupling agent may be supported on a polymer. For example, N-(3-dimethylaminopropyl)-N′-(ethyl)carbodiimide (EDC) supported on a polymer with a degree of functionalization of 1.4 mmol of EDC per gram of polymer is sold by Fluka (code 09657).

The at least one compound bearing at least one amine function may be present (or each compound may be present when there is more than one) in the presently disclosed composition, for example, in an amount ranging from 0.1% to 60% by weight, such as from 0.2% to 40% by weight, for example, from 0.5% to 35% by weight relative to the total weight of the composition.

The at least one pigment may be present (or each pigment may be present when there is more than one) in the presently disclosed composition, for example, in an amount ranging from 0.05% to 80% by weight, such as from 0.1% to 60% by weight and, for example, from 0.25% to 40% by weight relative to the total weight of the composition.

The at least one chemical coupling agent may be present (or each coupling agent may be present where there is more than one) in the presently disclosed composition, for example, in an amount ranging from 0.05% to 30% by weight, such as from 0.1% to 20% by weight and, for example, from 0.25% to 10% by weight relative to the total weight of the composition.

The composition according to the present disclosure may also comprise at least one additional compound, which, in combination with the at least one chemical coupling agent, is capable of promoting the formation of amide bonds.

According to one embodiment of the invention, the at least one additional compound is a compound bearing at least one N-hydroxyl function.

Non-limiting examples of compounds bearing at least one N-hydroxyl function that may be mentioned include N-hydroxysuccinimide; and N-hydroxysulfosuccinimide.

In at least one embodiment, the at least one compound bearing at least one N-hydroxyl function also contains at least one polar group.

The at least one additional compound which, in combination with the at least one chemical coupling agent, is capable of promoting the formation of amide bonds may be present (or each additional compond may be present where there is more than one), for example, in an amount ranging from 0.05% to 30% by weight, such as from 0.1% to 20% by weight and, for example, from 0.25% to 10% by weight relative to the total weight of the composition.

The composition in accordance with the disclosure may furthermore comprise fillers, with the exception of silica.

As used herein, the term “fillers” should be understood as meaning colorless or white, mineral or synthetic, lamellar or non-lamellar particles. The fillers may be present in an amount ranging from 0 to 80% by weight, such as from 0.01% to 60% by weight and, for example, from 0.02% to 40% by weight relative to the total weight of the composition. Non-limiting mention may be made, for example, of talc, zinc stearate, kaolin, polyamide (Nylon®) (Orgasol from Atochem) powders, polyethylene powders, powders of tetrafluoroethylene polymers (Teflon®), starch, boron nitride, polymer microspheres such as those of polyvinylidene chloride/acrylonitrile, for instance Expancel (Nobel Industrie), acrylic acid copolymer microspheres (Polytrap® from the company Dow Coming), silicone resin microbeads (for example Tospearls® from Toshiba) and elastomeric organopoly-siloxanes.

The composition according to the disclosure may also contain at least one conventional cosmetic additive chosen from fixing polymers, thickeners, anionic, nonionic, cationic or amphoteric surfactants, oxidation dyes such as oxidation bases and couplers, direct dyes other than pigments, fragrances, preserving agents, conditioning agents, sunscreens, proteins, vitamins, provitamins, anionic, nonionic, cationic or amphoteric non-fixing polymers, mineral, plant or synthetic oils, ceramides, pseudoceramides, volatile or non-volatile, linear or cyclic, modified or unmodified silicones, pH regulators, oxidizing agents, reducing agents, inhibitors, catalysts and any other additive conventionally used in cosmetic compositions.

A process according to the present disclosure comprises, for example, applying to keratin fibers:
at least one compound bearing at least one amine function as defined above;
at least one pigment as defined above; and
at least one chemical coupling agent as defined above.

The at least one compound bearing at least one amine function, the at least one pigment and the at least one chemical coupling agent may be applied to the keratin fibers using several compositions, each containing one or two of the at least one compound bearing at least one amine function, the at least one pigment, and the at least one chemical coupling agent, or using only one composition containing the at least one compound bearing at least one amine function, the at least one pigment and the at least one chemical coupling agent.

According to one embodiment of the present disclosure, a composition (A) comprising, in a cosmetically acceptable medium, the at least one compound bearing at least one amine function, the at least one pigment and the at least one chemical coupling agent, is applied to the keratin fibers.

According to another embodiment, a composition (B) comprising, in a cosmetically acceptable medium, the at least one compound bearing at least one amine function, and a composition (C) comprising, in a cosmetically acceptable medium, the at least one pigments and the at least one chemical coupling agent, is applied to the keratin fibers, the order of application of the compositions (B) and (C) being irrelevant.

According to another embodiment, a composition (B) comprising, in a cosmetically acceptable medium, the at least one compound bearing at least one amine function, a composition (D) comprising, in a cosmetically acceptable medium, the at least one pigment and a composition (E) comprising, in a cosmetically acceptable medium, the at least one chemical coupling agent, is applied to the keratin fibers, the order of application of the compositions (B), (D) and (E) being irrelevant.

According to another embodiment of the disclosure, a composition (F) comprising, in a cosmetically acceptable medium, the at least one compound bearing at least one amine function and the at least one chemical coupling agent and a composition (D) comprising, in a cosmetically acceptable medium, the at least one pigment, is applied to the keratin fibers, the order of application of the compositions (F) and (D) being irrelevant.

According to another embodiment of the disclosure, a composition (E) comprising, in a cosmetically acceptable medium, the at least one chemical coupling agent and a composition (G) comprising, in a cosmetically acceptable medium, the at least one compound bearing at least one amine function and the at least one pigment, is applied to the keratin fibers, the order of application of the compositions (E) and (G) being irrelevant.

According to one embodiment of the disclosure, the composition comprising the at least one pigment is applied before the composition(s)comprising the at least one compound bearing at least one amine function and/or the at least one chemical coupling agent.

According to one embodiment of the disclosure, at least one additional compound, which, in combination with the at least one chemical coupling agent, is capable of promoting the formation of amide bonds, as defined above, is applied to the keratin fibers to promote the formation of amide bonds.

For example, the at least one additional compound may be present in one or other or in several of the compositions applied to the keratin fibers or in an additional composition, in which case the order of application of the various compositions to the keratin fibers is irrelevant.

According to one embodiment of the disclosure, the pigment(s) are dispersed in the various compositions used in the process comprising them.

In these compositions, the at least one compound bearing at least one amine function is present (or each is present when there is more than one), for example, in an amount ranging from 0.1% to 60% by weight, such as from 0.2% to 40% by weight and, for example, from 0.5% to 35% by weight relative to the total weight of the composition comprising them; the at least one pigment is present (or each pigment is present when there is more than one), for example, in an amount ranging from 0.05% to 80% by weight, such as from 0.1% to 60% by weight and, for example, from 0.25% to 40% by weight relative to the total weight of the composition comprising them, the at least one chemical coupling agent is present (or each agent is present when there is more than one), for example, in an amount ranging from 0.05% to 30% by weight, such as from 0.1% to 20% by weight and, for example, from 0.25% to 10% by weight relative to the total weight of the composition comprising them.

These compositions may also contain various conventional cosmetic additives as defined above.

The various compositions used in the process in accordance with the present disclosure may be applied to dry or wet hair.

Intermediate rinsing and/or drying may be performed between each application. The lock may be dried under a hood, with a hairdryer or with smoothing tongs.

According to the same processes, it is possible to produce multiple superpositions of layers of polymers that crosslink together to achieve the desired type of deposit (in terms of chemical nature, mechanical strength, thickness, appearance, feel, etc.).

In the context of the present disclosure, a cosmetically acceptable medium generally comprises water and/or at least one cosmetically acceptable solvent chosen from, for example, alcohols, esters, ketones or cyclic volatile silicones or water-solvent(s) mixtures, these solvents in one embodiment, being C₁-C₄ alcohols.

A subject of the present disclosure is also a multi-compartment device comprising at least two compositions such that the combination of the compositions comprises:
at least one compound bearing at least one amine function as defined above;
at least one pigment as defined above; and
at least one chemical coupling agent as defined above.

According to one embodiment of the disclosure, a first compartment comprises the composition (B) as defined above and a second compartment comprises the composition (C) as defined above.

According to another embodiment, a first compartment comprises the composition (B) as defined above, a second compartment comprises the composition (D) as defined above and a third compartment comprises the composition (E) as defined above.

According to another embodiment of the disclosure, a first compartment comprises the composition (F) as defined above and a second compartment comprises the composition (D) as defined above.

According to another embodiment, a first compartment comprises the composition (E) as defined above and a second compartment comprises the composition (G) as defined above.

According to one embodiment of the disclosure, the combination of the compositions also comprises at least one additional compound, which, in combination with the at least one chemical coupling agent, is capable of promoting the formation of amide bonds, as defined above.

For example, one or other or several of the compositions contained in the multi-compartment device may also comprise the at least one additional compound.

The multi-compartment device may also comprisesan additional composition comprising, in a cosmetically acceptable medium, the at least one additional compound.

A subject of the present disclosure is also the use for dyeing keratin fibers of at least one compound bearing at least one amine function as defined above, at least one pigment as defined above and at least one chemical coupling agent as defined above.

In particular, one subject of the present disclosure is the use for dyeing keratin fibers of at least one compound bearing at least one amine function as defined above, at least one pigment as defined above and at least one chemical coupling agent as defined above, in which the coloration of the keratin fibers is shampoo-fast.

For example, in at least one embodiment, the shampoo-fastness is such that the percentage of degradation after six shampoo washes as defined below is less than 50%.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements.

The present disclosure will be illustrated in more detail with the aid of the non-limiting examples that follow.

### EXAMPLES

The following examples were performed using locks of 1 g of natural hair.

The various reagents used are as follows:
Pigment 1: Composite pigment of 17 nm with a silica core and an organic part consisting of quinacridone (pigment Red 122) prepared via the method described in patent application EP 1 184 426 A2;
Pigment 2: Calcium salt of lithol B red, D & C Red 7 sold by the company Wackherr;
EDC: N-(3-Dimethylaminopropyl)(N′-ethyl)carbodiimide hydrochloride sold by Fluka;
EDC supported on a polymer sold by Fluka (code 09657) with a degree of functionalization of 1.4 mmol of EDC per gram of polymer;
NHS: Sodium N-hydroxysulfosuccinimide sold by Fluka.

The following solutions were prepared from these reagents:
Solution A₁: Aqueous 10% solution of pigment 1;
Solution A₂: Aqueous 10% solution of pigment 2;
Solution B: Aqueous 10% solution of polyvinylamine/n-vinylformamide (Catiofast VFH sold by BASF);
Solution C: Aqueous solution containing 0.023 g of EDC and 0.026 g of NHS;
Solution D₁: Aqueous solution containing 10% of pigment 1 and 10% of polvinylamine/n-vinylformamide (Catiofast VFH sold by BASF);
Solution D₂: Aqueous solution containing 10% of pigment 2 and 10% of polyvinylamine/n-vinylformamide (Catiofast VFH sold by BASF);
Solution E: Aqueous solution containing 0.085 g of EDC supported on a polymer and 0.026 g of NHS.

### Example 1

1) 0.5 g of solution A₁ was applied to a lock of clean, dry natural hair. The lock was then dried under a hood.

2) 0.5 g of solution B was then applied to the dry lock. Without intermediate drying, 0.3 g of solution C was applied.

3) The lock was then placed under a hood for 30 minutes.

4) The lock was rinsed and shampooed.

The same procedure was performed for solution A₂.

With solution A₁, the lock obtained was strongly colored pink. With solution A₂, the lock obtained was strongly colored red. The locks were then subjected to a shampoo-fastness test.

The color of the hair was measured using a Minolta CM3600d® spectrocolorimeter in the CIELab system. In this system, L* represents the luminance, a* the hue and b* the saturation.

The degradation of the color after six shampoo washes was estimated according to the following formula:

$$\% degradation = 100 * \frac{DE6 Shamp}{DE Contr}$$

$$with DE contr = \\ \sqrt{{( {a}^{*} contr - {a}^{*} BN )}^{2} + {( {b}^{*} contr - {b}^{*} BN )}^{2} + {( {L}^{*} contr - {L}^{*} BN )}^{2}}$$

$$and DE 6 Shamp = \\ \sqrt{{( {a}^{*} contr - {a}^{*} 6 Sh )}^{2} + {( {b}^{*} contr - {b}^{*} 6 Sh )}^{2} + {( {L}^{*} contr - {L}^{*} 6 Sh )}^{2}}$$

in which a*BN, b*BN and L*BN are the values of a*, b* and L* for the uncolored lock, a*tem, b*tem and L*contr the values of a*, b* and L* for the colored lock before shampooing, while a*6Sh, b*6Sh and L*6Sh are the values of a*, b* and L* for the colored lock after 6 shampoo washes.

With solution A₁, the degradation of the color was 7.91% after 6 shampoo washes.

With solution A₂, the degradation of the color was 26.92% after 6 shampoo washes.

These results show that the coloration obtained with the process in accordance with the invention showed good shampoo-fastness.

### Example 2

1) 0.5 g of solution D₁ was applied to clean dry hair. Without intermediate drying, 0.3 g of solution C was applied.

2) The lock was then placed under a hood for 30 minutes.

3) The lock was rinsed and shampooed.

The same procedure was performed for solution D₂.

With solutions D₁, the lock obtained was strongly colored pink. With solution D₂, the lock obtained was strongly colored red.

### Example 3

1) 0.5 g of solution A₁ was applied to clean dry hair. The lock was then dried under a hood.

2) 0.5 g of solution B was then applied to the dry lock. Without intermediate drying, 0.3 g of solution C was applied.

3) The lock was then dried with smoothing tongs at 180° C.

4) The lock was rinsed and shampooed.

The same procedure was performed for solution A₂.

With solution A₁, the lock obtained was strongly colored pink. With solution A₂, the lock obtained was strongly colored red.

### Example 4

1) 0.5 g of solution A₁ was applied to clean dry hair. The lock was then dried under a hood.

2) 0.3 g of solution C was then applied to the dry lock. Without intermediate drying, 0.5 g of solution B was applied.

3) The lock was then placed under a hood for 30 minutes.

4) The lock was rinsed and shampooed.

The same procedure was performed for solution A₂.

With solution A₁, the lock obtained was strongly colored pink. With solution A₂, the lock obtained was strongly colored red.

### Example 5

1) 0.5 g of solution A₁ was applied to clean dry hair. The lock was then dried under a hood.

2) 0.5 g of solution B was then applied to the dry lock. Without intermediate drying, 0.3 g of solution E was applied.

3) The lock was then placed under a hood for 30 minutes.

4) The lock was rinsed and shampooed.

The same procedure was performed for solution A₂.

With solution A₁, the lock obtained was strongly colored pink. With solution A₂, the lock obtained was strongly colored red.

Results comparable to those outlined above were obtained with the following polyamines:
ε-polylysine (sold by Chisso);
polyethyleneimine (Lupasol P sold by BASF);
poly(allylamine hydrochloride) (sold by Aldrich, code 28322-3).

1. A composition for dyeing keratin fibers, comprising, in a cosmetically acceptable medium:
at least one compound bearing at least one amine function;
at least one pigment other than iron oxide, titanium oxide and silica; and
at least one chemical coupling agent for the formation of amide bonds.

2. A composition according to claim 1, wherein the at least one compound bearing at least one amine function has a molecular weight of greater than 300 g/mol⁻¹.

3. A composition according to claim 2, wherein the at least one compound bearing at least one amine function has a molecular weight of greater than 1000 g.mol⁻¹.

4. A composition according to claim 2, wherein the at least one compound bearing at least one amine function is chosen from polymers.

5. A composition according to claim 4, wherein the polymers are polyamines.

6. A composition according to claim 5, wherein the polyamines are chosen from polyvinylamine, polyethyleneamine, polylysine, chitosan, polyallylamine, aminodextran, aminocellulose and aminopolyvinyl acetal.

7. A composition according to claim 1, wherein the at least one pigment is in the form of powder or of pigmentary paste.

8. A composition according to claim 7, wherein the at least one pigment is a mineral pigment chosen from zirconium oxides, cerium oxides, chromium oxides, manganese violet, ultramarine blue, chromium hydrate and ferric blue.

9. A composition according to claim 7, wherein the at least one pigment is an organic pigment chosen from nitroso, nitro, azo, xanthene, quinoline, anthraquinone and phthalocyanin compounds, metallic complex compounds, and isoindolinone, isoindoline, quinacridone, perinone, perylene, diketopyrrolopyrrole, thioindigo, dioxazine, triphenylmethane and quinophthalone compounds.

10. A composition according to claim 7, wherein the at least one pigment is a composite pigment comprising particles comprising:
at least one mineral core,
at least one binder for fixing the pigments to the mineral core, and
at least one organic pigment at least partially covering the mineral core.

11. A composition according to claim 7, wherein the at least one pigment is a lake comprising a mineral substrate chosen from alumina, silica, calcium sodium borosilicate, calcium aluminium borosilicate, and aluminium, owherein a dye is absorbed on the mineral substrate.

12. A composition according to claim 7, wherein the at least one pigment is a pigment with special effects chosen from nacreous pigments, pigments with interference effects not bound to a substrate, fluorescent pigments, phosphorescent pigments, photochromic pigments, thermochromic pigments and quantum dots.

13. A composition according to claim 12, wherein the nacreous pigments are chosen from mica coated with titanium or with bismuth oxychloride, mica coated with titanium and with iron oxides, mica coated with titanium and with ferric blue or chromium oxide, mica coated with titanium and with an organic pigment chosen from nitroso, nitro, azo, xanthene, quinoline, anthraquinone and phthalocyanin compounds, metallic complex compounds, and isoindolinone, isoindoline, quinacridone, perinone, perylene, diketopyrrolopyrrole, thioindigo, dioxazine, triphenylmethane and quinophthalone compounds, and nacreous pigments based on bismuth oxychloride.

14. A composition according to claim 12, wherein the pigments with an interference effect not bound to a substrate are chosen from liquid crystals and holographic interference flakes.

15. A composition according to claim 1, wherein the at least one pigment is chosen from colored pigments.

16. A composition according to claim 1, wherein the at least one pigment is in a dispersion.

17. A composition according to claim 1, wherein the at least one chemical coupling agent is chosen from carbodiimide derivatives; 1,1′-carbonyldiimidazole; phosphonium salts; phosphonates; phosphoramides; and dialkoxytriazine derivatives.

18. A composition according to claim 17, wherein the carbodiimide derivatives are chosen from the compounds of formula (I):
R₁—N═C═N—R₂ (I)
wherein R₁ and R₂ are chosen from, independently of each other:
a hydrogen atom;
alkyl radicals, which are unsubstituted or substituted with at least one substituent chosen from:
a hydroxyl radical;
an amino radical;
monoalkylamino an dialkylamino radicals;
mono- ans di(mono- or polyhydroxyalkyl)amino radicals;
aryl radicals;
heterocyclic groups;
aryl radicals;
arylalkyl radicals.

19. A composition according to claim 18, wherein neither R₁ nor R₂ is a hydrogen atom.

20. A composition according to claim 19, wherein neither R₁ nor R₂ is chosen from unsubstituted or substituted alkyl radicals.

21. A composition according to claim 17, wherein the carbodiimide derivatives are chosen from N-(3-dimethylaminopropyl)-N′-(ethyl)carbodiimide; and N,N′-dicyclohexylcarbodiimide.

22. A composition according to claim 17, wherein the carbodiimide derivatives are soluble in water or in an alcoholic solvent.

23. A composition according to claim 1, wherein the each compound bearing at least one amine function is present in an amount ranging from 0.1% to 60% by weight relative to the total weight of the composition.

24. A composition according to claim 1, wherein each pigment is present in an amount ranging from 0.05% to 80% by weight relative to the total weight of the composition.

25. A composition according to claim 1, wherein each chemical coupling agent is present in an amount ranging from 0.05% to 30% by weight relative to the total weight of the composition.

26. A composition according to claim 1, comprising at least one additional compound, which, in combination with the at least one chemical coupling agent, is capable of promoting the formation of amide bonds.

27. A composition according to claim 26, wherein the at least one additional compound is chosen from compounds bearing at least one N-hydroxyl function.

28. A composition according to claim 27, wherein the compounds bearing at least one N-hydroxyl function are chosen from N-hydroxysuccinimide; and N-hydroxysulfosuccinimide.

29. A composition according to claim 27, wherein the compounds bearing at least one N-hydroxyl function also contain at least one polar group.

30. A composition according to claim 26, wherein each additional compound is present in an amount ranging from 0.05% to 30% by weight relative to the total weight of the composition.

31. A process for dyeing keratin fibers, comprising applying the following compounds to the keratin fibers:
(a) at least one compound bearing at least one amine function;
(b) at least one pigment other than iron oxide, titanium oxide, and silica; and
(c) at least one chemical coupling agent for the formation of amide bonds.

32. A process according to claim 31, wherein compounds (a), (b), and (c) are applied to the keratin fibers using several compositions each containing one or two of (a), (b), and (c), or using only one composition containing all three compounds (a), (b), and (c).

33. A process according to claim 32, wherein the composition comprising (b) is applied before the composition(s) comprising (a) and/or (c).

34. A process according to claim 31, wherein at least one additional compound which, in combination with the at least one chemical coupling agent, is capable of promoting the formation of amide bonds, is applied to the keratin fibers to promote the formation of amide bonds.

35. A multi-compartment device comprising at least two compositions such that the combination of the compositions comprises:
at least one compound bearing at least one amine function;
at least one pigment other than iron oxide, titanium oxide, and silica; and
at least one chemical coupling agent for the formation of amide bonds.

36. A multi-compartment device according to claim 35, wherein the combination of compositions also comprises at least one additional compound which, in combination with the at least one chemical coupling agent, is capable of promoting the formation of amide bonds.

37. A process for rendering coloration of keratin fibers shampoo-fast, comprising applying to the keratin fibers:
(a) at least one compound bearing at least one amine function;
(b) at least one pigment other than iron oxide, titanium oxide, and silica; and
(c) at least one chemical coupling agent for the formation of amide bonds.
